(19) United States
(12) Patent Application Publication
(10) Pub. No.: US 2024/0312586 A1
(43) Pub. Date: Sep. 19, 2024

(54) SYSTEMS, METHODS AND COMPUTER-ACCESSIBLE MEDIUM FACILITATING DEPLOYMENT OF ANTIDEPRESSANTS AND OTHER PRESCRIPTION DRUGS

(71) Applicant: New York University, New York, NY (US)

(72) Inventors: ANDRE FENTON, New York, NY (US); ALEXANDER BOROVIK, Munich (DE); NICHOLAS MASON, London (GB)

(21) Appl. No.: 18/575,905

(22) PCT Filed: Jun. 29, 2022
(86) PCT No.: PCT/US2022/035503

§ 371 Date: Jan. 2, 2024

Related U.S. Application Data
(63) Continuation of application No. PCT/US2022/035503, filed on Jun. 29, 2022.
(60) Provisional application No. 63/216,654, filed on Jun. 30, 2021.

(51) Int. Cl. G16H 20/10; G16H 10/60; G16H 80/00

(52) U.S. Cl. CPC G16H 20/10; G16H 10/60; G16H 80/00

(57) ABSTRACT

Computer-accessible mediums, computer implemented methods and systems can be provided for producing, distributing, electronically prescribing and administering prescription drug medication, and medical procedures e.g., using independent verifiable Self-Sovereign Identity (SSI) credentials. For example, it is possible to provide one or more prescription contracts associated with a medical professional to a mobile storage configuration of a patient via a first digital communication. In addition, it is possible to submit a co-execution of the one or more prescription contracts by the patient to a control application of the medical professional via a second electronic communication. Further, it is possible to provide one or more prescriptions associated with the prescription contract(s) by the patient for a medical product, a device and/or a medication for a validation via a third electronic communication. In the first, second, and third communications, credentials of at least one of the patient, the medical professional, or the dispensary receiving the one or more prescription contracts or the one or more prescriptions can be retrieved and verified using (i) the SSI credentials and (ii) a database (e.g., an issuer database and/or a verifier database).

### CROSS-REFERENCE TO RELATED APPLICATION(S)

This application relates to and claims priority from U.S. Patent Application No. 63/216,654, filed on Jun. 30, 2021, the entire disclosure of which is incorporated herein by reference.

### FIELD OF THE DISCLOSURE

The present disclosure related generally to systems, methods and computer-accessible mediums for facilitating prescriptions and administration of drug medication(s) on a peer-to-peer network, e.g., for preventing prescription drug abuse, including via interactions such as the co-signing of a prescription contract, the issuance and acceptance of a patient program, and the verification of adherence to that patient program, facilitated by a web dashboard and mobile application for exchanging, viewing and monitoring verifiable health and non-health related information between the stakeholders.

### BACKGROUND INFORMATION

Prescription drug abuse and addiction continues to plague many individuals and their families across the world. Further, because of prescription drug abuse and related personal health and social issues, bringing new classes of prescribed drugs to the market is challenging for the pharmaceutical market. The responsible use of prescription medication by patients is often determined by the ability of physicians to personally prescribe, administer and monitor prescribed drug use through regular face-to-face interactions. This requirement for face-to-face interactions can even constrain the chemistry and what medications and doses are feasible as in the example case that the medication administered in a face-to-face interaction has to be effective for 1 week until the next interaction, whereas an effective medication that can be taken twice a day at home as a short-acting pill would not be viable if the face-to-face interaction is mandatory.

Accordingly, there is a need to provide systems, methods and computer accessible mediums for availing a prescription and administration of controlled medication and procedures which, for example, facilitate the recording of data to allow new, and safer, prescription medications and prescription medication practices to be developed, and which also may be used to reduce patients' ability to abuse their prescribed medications.

Further, the vaccines-for-data trade-off between Pfizer and the government of Israel has sparked impassioned debate among data privacy experts, biotech researchers and medical ethics boards, weighing the potential benefits of mining the population for vaccine and drug use insights against the potential abuse of millions of personal medical records. This demonstrates that pharma has interest in getting access to patient meta data and there may be major public interest in sharing such data with pharma; however, the mechanisms or technology for collecting and sharing data are not developed.

Thus, there may be a need to address and/or at least partially overcome at least some of the prior deficiencies described herein by providing, e.g., systems, methods and computer-accessible mediums which facilitate data sharing with pharmaceutical companies, e.g., while preserving patients' privacy.

### SUMMARY OF EXEMPLARY EMBODIMENT(S)

Exemplary embodiments of the present disclosure described herein can address such needs by providing systems, methods and computer-accessible mediums which facilitate a remote prescription and administering of prescription medication, and providing at home use by a patient without creating a risk of the patient using the prescription medicine in a way not intended by the prescribing physician.

These and many more advantages are provided in the exemplary embodiments of the present disclosure.

Exemplary embodiments of the systems, methods and computer-accessible mediums described herein can include a computer implemented method for prescribing and/or administering prescription drug medication in a Self-Sovereign Identity (SSI) network supporting peer-to-peer electronic communications using verifiable credentials defined as schemas. Verifiable credentials can be stored in the issuers/verifier databases, managed from their web dashboard(s), and patient mobile application(s). No personal or health information is stored in a central database, which can provide a high level of patient and physician data privacy and security. Such exemplary systems, methods and computer-accessible mediums can issue a prescription contract from a physician from their web dashboard, including a digital signature of the physician, to the patient's mobile wallet via a first electronic communication. It is possible to verify and co-sign such prescription contract by the patient via a second electronic communication from the patient to the physician; and receiving and verifying the prescription contract by a pharmacy from a third electronic communication from the patient. The exemplary systems, methods and computer-accessible mediums may be used to submit a patient program in a fourth electronic communication, including a digital schedule defining procedures for a submission of record of use of the prescription medication by the patient, from the physician web dashboard to the patient mobile application; and submitting a record of a use of the prescription medication by the patient in the patient's mobile wallet to the physician in the web dashboard or another third-party via a fifth electronic communication. The exemplary systems, methods and computer-accessible mediums can further facilitate an alteration of the prescription contract and submitting the altered prescription contract for co-signing from the physician web dashboard to the patient mobile application via a sixth electronic communication. In addition, the exemplary systems, methods and computer-accessible mediums can further facilitate an alteration of the patient program and submitting the altered patient program from the physician web dashboard to the patient mobile application in a seventh electronic communication. Further, the exemplary systems, methods and computer-accessible mediums can also facilitate an issuance of a notice of patient program compliance and prescription contract fulfilment from the physician web dashboard to the patient mobile application in an eighth electronic communication. In addition, the exemplary systems, methods and computer-accessible mediums can additionally facilitate a renewal of the prescription contract and repeating the cycle of interactions in additional electronic communications between the entities. Each of the electronic communications in the exemplary method can include credentials of the entity receiving data to be retrieved and verified using an issuer/verifier database by the entity transmitting data.

The exemplary systems, methods and computer-accessible mediums can further include and/or provide additional electronic communications between users of the web dashboard, including physicians, psychiatrists, guardians and other trusted parties, and users of the patient mobile application such as, but not limited to, identifying issuing and verifying licenses, qualifications and credentials issuance and verification; issuing and verifying health records; performing consent-related requests and actions; performing payment related requests and actions; adding new stakeholders; and peer-to-peer messaging, promotions and surveying.

According to further exemplary embodiments of the present disclosure, each pill in the prescription medication can include an individual scannable QR code, and the digital schedule can define exemplary procedures for a submission of record of use for each pill of the prescription medication. Further, the QR codes for each pill of the prescription medication can be accessible, e.g., only after a packaging is opened and each pill is removed from the packaging. In addition, at least one of the QR codes can be made to appear only temporarily through an existing or novel method such as by using ink that is sensitive to one of light or air so that the at least one of the QR codes would fade once the packaging reveals such one or more of the QR codes is opened; and/or the QR code can be concealed on a printed electrochromic display and activated to appear temporarily by placing a near field communication device, such as a mobile phone, e.g., near the display. Further, the submission of record of use by the fourth interaction can include one or more of a geography location, a timestamp, a photograph, a third-party attestation and other metadata.

According to yet further exemplary embodiments of the present disclosure, the exemplary method can include presenting, via a peer-to-peer interaction by the patient, one or more of the prescription contracts, patient programs, records of use, or other health or non-health related data to a third party using a zero-knowledge proof, whether by selective disclosure or full disclosure. The exemplary systems, methods and computer-accessible mediums can be implemented in or as a SSI network, in which the physician and other individual or institutional stakeholders can also be easily added to the SSI network and credentialed using a decentralized public key infrastructure. According to still further exemplary embodiments of the present disclosure, an additional procedure may be added for confirming pills in the prescription are being consumed by adding one of several tastes or flavors to the medications such as, for example sweetness, sourness, saltiness, or bitterness. The subject may need to declare in the corresponding patient mobile application which taste corresponds to the pill, each of which has a corresponding QR code. Matching the taste to the QR code can be taken as evidence the particular pill was consumed. If, for example, the pill could have one of four tastes, then there may be, e.g., only a 25% chance the patient could guess the correct flavor. The timing (and geolocation and other meta data) of the user's response would be expected to be proximate in time to the QR code scan, if the pills are being consumed legitimately. According to further exemplary embodiments, for the exemplary systems, methods and computer-accessible mediums can further facilitate a confirmation that the pills in the prescription medication are being consumed by using a small amount of an ingestible dye with a transient color that causes the tongue to change in color for a duration of time. The ingestible dye may be one of a blue-colored fluorescent protein Phycocyanin, a Bismuth subsalicylate (which causes the tongue to appear black), commonly used food colorings, such as those commonly used in commercial candy and popsicle products, Green Fluorescent Protein (“GFP”), Red Fluorescent Protein (RFP), Blue Florescent Protein (“BFP”), or the like. The ingestible dye may include a marker peptide which split in two halves, a first halve may be provided in a first pill, and a second halve may be provided in a second pill, and the marker peptide would only be reconstituted if both the first and second pills are consumed by the patient.

Thus, e.g., a patient's tongue may appear to change color if both the first and second pills are ingested by a patient over a certain duration of time, and would not change color if only one of the first or second pills is ingested by itself. The peptide components may also be observed visually (and temporarily) only in the presence of incident light of the appropriate activation spectra. Exemplary embodiments described herein may detect the color by machine-intelligent algorithmic comparison of a before-pill consumption and an after-pill consumption digital visual image of the patient's tongue. The timing (and geolocation and other meta data) of the digital image captures would be expected to be proximate in time to the QR code scan, if the pills are being consumed legitimately.

As an example, if a patient takes the pill on Monday, there may be a specific taste or specific tongue-color associated with the day's pill. Taking a pill on the next occasion (for example, on Tuesday) can produce a different outcome (taste or color) because those pills are tagged by a different taste or color. If each pill has a particular taste or color and QR code then these pill components would serve as a validation system. The patient mobile application's assessment of tongue colors, record of the user's taste perception, and/or other determining qualities could be recorded, timestamped and verified against the information transmitted through the QR code to produce a confirmation that the medication has or has not been ingested in compliance with the digital schedule.

According to further exemplary embodiments of the present disclosure, an additional procedure can be added for a patient to receive two pills in combination. A first pill can have the active compound, such as MDMA or ketamine with a chemical modification that makes it inert. The first pill may be considered a “lock”. The second pill can serve as a “key” that interacts with the “lock” to release the ketamine/MDMA. By providing a variety of potential locks and keys a doctor or other patient healthcare manager can use the mobile application to provide corresponding locks and keys for prescription drugs to be effective.

According to additional exemplary embodiments of the present disclosure, the exemplary systems, methods and computer-accessible mediums can further facilitate providing access, submission, verification, monitoring, analysis, reporting and adjustment of the prescription contracts, the record of use, the patient program and any other data by a physician, psychiatrist, pharmacy and other authorized third parties through a web dashboard.

According to further exemplary embodiments of the present disclosure, the exemplary systems, methods and computer-accessible mediums can further facilitate a determination of the risk of a particular patient abusing their prescription or not adhering to their patient program by a physician, psychiatrist or other authorized party to using a rules-based or machine learning component. The data input for such a component may be drawn from the indicated exemplary methods and/or other possible public and private sources.

According to yet further exemplary embodiments of the present disclosure, the exemplary systems, methods and computer-accessible mediums can further facilitate a generation of insights and recommendations that would assist a physician, psychiatrist or other authorized party to take certain interventions using a rules-based or artificial intelligence component.

According to still further exemplary embodiments of the present disclosure, the exemplary systems, methods and computer-accessible mediums can further facilitate an assistance for different stakeholders with healthcare, commercial, regulatory and other needs through an automated or manual process and on a one-time or periodic basis using a reporting mechanism in the web dashboard. For example, the same and similar mechanisms and interactions as described above for patient—caregiver interactions can be used amongst growers and manufacturers, certifying laboratories and transport providers to establish chain of custody and quality certifications for nutraceutical, bioceutical and pharmaceutical products, as part of the process of providing care to the patient population that is being treated. As a further example, after the patient has received a particular treatment and as part of the ongoing treatment program, the system described herein can be used to provide and facilitate ongoing patient—caregiver interactions that provide for therapeutic monitoring and support as needed or wanted via private structured and verifiable interactions.

According to an additional exemplary embodiment of the present disclosure, methods, systems and computer-accessible mediums can be provided to perform these exemplary functions and/or procedures described herein, e.g., using the indicated exemplary interactions and arrangements described herein.

According to a further exemplary embodiment of the present disclosure, an SSI system, method and computer-accessible medium for creating, sharing and co-signing prescription contracts and patient programs between physicians and patients can be provided. The exemplary systems, methods and computer-accessible mediums can facilitate a physician to electronically issue a prescription contract and/or patient program to a patient, and for a patient to electronically verify and sign the contract and/or the contents of the patient program. The exemplary systems, methods and computer-accessible mediums can facilitate electronically changing the prescription contract and patient program and recording the patient's verification and acceptance of those changes. Further, the exemplary systems, methods and computer-accessible mediums can facilitate the electronic approval and verification fulfilment of, cancellation or renewal of a prescription contract and/or patient program.

According to still another additional exemplary embodiment of the present disclosure, an SSI system, method and computer-accessible medium for the electronic verification of prescription compliance using independent verifiable credentials can be provided. The exemplary systems, methods and computer-accessible mediums can facilitate the electronic and remote verification of a pill or other medication was consumed in a timely manner using a taste survey.

According to yet another exemplary embodiment of the present disclosure, an SSI exemplary systems, methods and computer-accessible mediums for electronic verification of prescription compliance using aggregated independent verifiable credentials can be provided. The exemplary systems, methods and computer-accessible mediums can facilitate an electronic verification that a pill was consumed in a timely manner using two or more of SSI verification of a taste or color survey and/or a QR-scan event and/or a proximity event and/or a timestamping event and/or a photograph and/or a third-party attestation using a user's mobile device.

According to an additional exemplary embodiment of the present disclosure, an SSI exemplary systems, methods and computer-accessible mediums for health professionals to send health-related reminders, notifications, messages and promotions to patients can be provided. The exemplary systems, methods and computer-accessible mediums can facilitate the manual or automatic transmission of text notifications or calls to remind a patient to take their medication if a medication registration event has not occurred in a timely manner.

According to another exemplary embodiment of the present disclosure, an SSI exemplary systems, methods and computer-accessible mediums that analyses data input to generate a patient risk classification and treatment recommendations can be provided. The exemplary systems, methods and computer-accessible mediums can analyze anonymized patient data to determine the patient's propensity to abuse a drug or fail to adhere or comply with their prescription program.

According to an additional exemplary embodiment, an exemplary SSI systems, methods and computer-accessible mediums for delegation of authority from a patient to a helper/guardian to act on their behalf in medical interactions and transactions can be provided. The exemplary systems, methods and computer-accessible mediums can facilitate an individual known to the patient to act on the patient's behalf in a delegated manner to organize the patient's appointments with authorized health professionals, share health-related symptoms and updates with those health professionals, order and collect the patient's prescriptions and fulfil the patient's prescription compliance requirements. The exemplary systems, methods and computer-accessible mediums can further facilitate an individual known to the patient to access, see, use, store and process health data relating to the patient's record, prescriptions, prescription compliance and adherence, non-prescription medications, symptoms and other formal and informal health related interactions and data. In addition, the exemplary systems, methods and computer-accessible mediums can facilitate an individual known to the patient to carry out an intervention for the patient on the advice of the physician or other health professional at some point in the future.

According to an additional exemplary embodiment, an exemplary systems, methods and computer-accessible mediums facilitating patients, health professionals and other human stakeholders involved in the patient's health care to electronically and remotely verify their own and each other's identity and professional accreditation via an SSI network can be provided. The exemplary systems, methods and computer-accessible mediums can facilitate a patient to electronically verify the identity and professional accreditation and credentials of a physician or health professional using SSI and verifiable credentials for remote healthcare services such as telemedicine. In addition, the exemplary systems, methods and computer-accessible mediums can facilitate a patient to electronically verify the identity and professional accreditation and credentials of a physician or health professional using SSI and verifiable credentials prior to that health professional entering the home or property of the patient. The exemplary systems, methods and computer-accessible mediums can also facilitate a patient to electronically submit a verifiable credential of their own identity in order to book, pay for and check-in to an appointment with a health professional. Further, the exemplary systems, methods and computer-accessible mediums can facilitate a physician to electronically verify the identity, address, insurance details and health-related information of a patient during patient on-boarding in a remote interaction using SSI. The exemplary systems, methods and computer-accessible mediums can also facilitate a pharmacist to electronically verify the identity of the customer matches the identity on the prescription using SSI.

According to an additional exemplary embodiment, an exemplary SSI systems, methods and computer-accessible mediums for facilitating patients and health professionals to electronically provide, share, record and recollect their consent for certain health interactions can be provided. The exemplary systems, methods and computer-accessible mediums can facilitate the organization of consent for the electronic exchange of anonymized or de-anonymized health data for prescriptions. In addition, the exemplary systems, methods and computer-accessible mediums can organize consent for the electronic use of anonymized health data in machine learning and artificial intelligence development and analysis.

According to an additional exemplary embodiment, an exemplary SSI systems, methods and computer-accessible mediums for adding health and non-health related stakeholders to the patient's care program can be provided. The exemplary systems, methods and computer-accessible mediums can electronically add therapists, nutritionists, fitness professionals, wellness professionals, education professionals, administrators, and/or their systems to a patient's program and each party can interact with the patient electronically using verifiable credentials in the SSI platform.

According to an additional exemplary embodiment, exemplary systems, methods and computer-accessible mediums for billing and invoicing health related activities based on tracking of verifiable credentials in an SSI platform can be provided. The exemplary systems, methods and computer-accessible mediums can facilitate the electronic administration, verification, and the paying of bills and invoices of clinical consultations, prescriptions and patient monitoring interactions based on the tracking of anonymized verifiable credential interactions in an SSI platform.

According to an additional exemplary embodiment, an exemplary SSI systems, methods and computer-accessible mediums for electronically scoring a patient's propensity to adhere to a prescription can be provided. The exemplary systems, methods and computer-accessible mediums can facilitate physicians and other health professionals to electronically contribute to and electronically verify the extent a patient has complied with and adhered to its patient program based on the patient's electronic interactions with health professionals.

According to an additional exemplary embodiment, an exemplary SSI systems, methods and computer-accessible mediums facilitating patients to electronically present their medical record to insurance companies using verifiable SSI credentials can be provided.

According to an additional exemplary embodiment, an exemplary systems, methods and computer-accessible mediums facilitating patients to electronically present their record of prescription adherence and compliance to insurance companies can be provided, so that those insurance companies can better serve the patient through a better care plan, better price or other adjustment.

According to an additional exemplary embodiment, an exemplary SSI systems, methods and computer-accessible mediums for facilitating patients to electronically compile, store, recollect, share and review medical records in verifiable credential format can be provided. The exemplary systems, methods and computer-accessible mediums can be integrated into electronic medical record systems and facilitate patients to electronically request and receive medical records using verifiable credentials. The records can be electronically compiled, stored, recollected, shared and reviewed by the patient and electronically provided to other healthcare stakeholders to check for prescription or patient program compliance and adherence.

According to an additional exemplary embodiment, an exemplary SSI systems, methods and computer-accessible mediums for physicians and care sites to electronically record and prove the month-on-month change in prescription adherence of their patients can be provided. The exemplary systems, methods and computer-accessible mediums can facilitate the electronic conversion of month-on-month records of prescription adherence and compliance into verifiable credentials that can be onward electronically provisioned to insurance and other stakeholders for demonstrating improved patient outcomes and reduced liability.

According to an additional exemplary embodiment, an exemplary SSI systems, methods and computer-accessible mediums for health professionals to electronically refer patients to other health professionals can be provided. The exemplary systems, methods and computer-accessible mediums can facilitate electronic health professional referrals using electronic recommendation attestation from the referrer, and the referee can electronically uphold or dispute the attestation based on the quality of care received. The exemplary systems, methods and computer-accessible mediums can facilitate electronic review of recommendation attestations by patients, and can provide financial remuneration to referrers who provide high quality referrals.

According to an additional exemplary embodiment, an exemplary SSI systems, methods and computer-accessible mediums for pharmacies to electronically verify patient prescriptions can be provided. The exemplary systems, methods and computer-accessible mediums can facilitate verification by pharmacies of patient prescriptions regardless of the number of prescription signatories (physicians) and medication types. The system can further facilitate pharmacies to electronically validate that the patient's prescription(s) when it has been fulfilled and electronically provide confirmation to the patient's prescribing physician(s) and that the patient program has commenced. The system can further facilitate the electronic fulfilment of prescription medication by the pharmacy, and electronic transmission of a confirmation to the patient and a notification of the commencement of the patient program. The notification can inform the patient that their compliance is being recorded per their prescription contract and patient program.

According to an additional exemplary embodiment, an exemplary SSI systems, methods and computer-accessible mediums for pharmacies to electronically generate follow-on sales growth from prescriptions can be provided. The exemplary systems, methods and computer-accessible mediums can facilitate electronic recommendation by physicians for non-prescription medicines for the patient to buy over the counter that the physician determine will effectively complement the patient's prescription.

According to an additional exemplary embodiment, an exemplary SSI systems, methods and computer-accessible mediums for facilitating the anonymization, aggregation, analysis and presentation of patient data can be provided. The exemplary systems, methods and computer-accessible mediums can facilitate pharmaceutical companies to electronically acquire anonymized and aggregated data pertaining to drug distribution, performance, adherence, compliance and the environmental and demographic conditions of patients consuming the drug is provided. The exemplary systems, methods and computer-accessible mediums can further facilitate insurance companies to electronically acquire anonymized and aggregated data on the compliance and adherence of patients to particular drugs, as well as the environmental and demographic conditions of patients. In addition, the exemplary systems, methods and computer-accessible mediums can facilitate other qualified researchers and stakeholders to electronically acquire anonymized and aggregated data on available data points. The exemplary systems, methods and computer-accessible mediums can also facilitate pharmaceutical companies to attribute increased sales of a particular drug to increased compliance and adherence to that drug.

According to an additional exemplary embodiment, an exemplary SSI systems, methods and computer-accessible mediums for electronically supporting continuity of care with accurate and verifiable medical records for a patient moving between places or institutions can be provided. The exemplary systems, methods and computer-accessible mediums can facilitate local (geographic or institutional) physicians to accelerate the issuance of prescriptions to non-local patients based on the verifiable (and possibly translated) prescription contract and patient program issued to the patient by a non-local physician and held by the patient in verifiable credential format.

According to an additional exemplary embodiment, an exemplary SSI systems, methods and computer-accessible mediums for facilitating physicians, pharmacists and patients to electronically authenticate the source, licensing, guidance, recommended use (etc.) of pharmaceutical products can be provided. The exemplary systems, methods and computer-accessible mediums can facilitate stakeholders to electronically scan a barcode on product packaging where that barcode triggers an action in the App to complete cryptographic verification of the verifiable credential associated with that product.

According to a still further exemplary embodiment of the present disclosure, computer-accessible mediums, computer implemented methods and systems can be provided for producing, distributing, electronically prescribing and administering prescription drug medication e.g., using independent verifiable Self-Sovereign Identity (SSI) credentials. For example, it is possible to provide one or more prescription contracts associated with a medical professional to a mobile storage configuration of a patient via a first digital communication. In addition, it is possible to submit a co-execution of the one or more prescription contracts by the patient to a control application of the medical professional via a second electronic communication. Further, it is possible to provide one or more prescriptions associated with the prescription contract(s) by the patient for a medical product, a device and/or a medication for a validation via a third electronic communication. In the first, second, and third communications, credentials of at least one of the patient, the medical professional, or the dispensary receiving the one or more prescription contracts or the one or more prescriptions can be retrieved and verified using (i) the SSI credentials and (ii) a database (e.g., an issuer database and/or a verifier database).

In yet another exemplary embodiment of the present disclosure, it is possible to submit an electronic program of the patient to the mobile storage configuration via a fourth communication. The electronic program can include a digital schedule defining procedures for the submission of an electronic record regarding use of the device, the medication and/or the product associated with the prescription by the patient. In addition, it is possible to submit the electronic record regarding use of the device, the medication and/or the product associated with the prescription by the patient to the medical professional via a fifth communication. For example, credentials of the patient and/or the medical professional who receive data can be retrieved and verified using the database via the fourth and fifth communications.

Alternatively or in addition, it is possible to modify the prescription contract(s), submit the modified prescription contract(s) by the medical professional to the mobile storage configuration of the patient via a sixth communication. An electronic program of the patient can be modified and submitted by the medical professional to the mobile storage configuration in a seventh communication. Credentials of the patient and/or the medical professional who receive data can be retrieved and verified using the database via the sixth communication.

Alternatively or in addition, it is possible to submit an electronic program of the patient to the mobile storage configuration, whereas the electronic program includes a digital schedule defining procedures for the submission of the electronic record regarding use of the medication that includes one or more pills associated with the prescription by the patient. For example, each of the pill(s) can include an individual scannable code, and the digital schedule can define procedures for a submission of the electronic record of use for each of the one or more pills. The scannable code for each of the one or more pills can be accessible, e.g., only after a packaging is opened and each of the one or more pills is removed from the packaging. The scannable code can be composed of an ink that is sensitive to at least one of light or air such that the scannable code fades when the packaging revealing the scannable code is opened.

For example, the packaging can contain a first pill and a second pill of the medication. The first pill can include a first type of ingestible dye, and the second pill can contain a second type of ingestible dye. A consumption of the first and second pills can cause a discoloration of a tongue of the patient over a particular time period. The submission of the electronic record associated with a use of the first and second pills can include a submission of a digital photograph of the tongue of the patient after each of the first and second pills is consumed. A compliance with the digital schedule can include monitoring of the tongue of the patient in submitted digital photographs. The packaging can contains a plurality of pills, and each of the pills can have a different taste from another one of the pills. The submission of the electronic record associated with a use of the pills can include a submission of the taste of each of the pills and a scannable code identifying a particular one of the pills ingested. A compliance with the digital schedule can include a determination of whether the identified taste matches the identified one of the pills.

According to still further exemplary embodiments of the present disclosure, the submission of the electronic record regarding the use can include a geography location, a timestamp, an identity of the patient, a photograph, a third-party attestation, and/or other meta data. It is also possible to electronically access one of the prescription contract(s) and the electronic program of the patient by a third party using a zero-knowledge proof.

Additionally or alternatively, it is possible to communicate with an electronic dashboard for providing access, submission, and verification of the prescription contract(s), the electronic record of use and/or the electronic program of the patient by at least one of the medical professional, the patient, the dispensary or an authorized third party via at least one computer processor. The retrieval and verification can occur in a decentralized public key infrastructure. Confidential information of the patient contained in the prescription contract(s), the electronic record associated with the use and/or the electronic program of the patient can be restricted from access.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a block diagram of exemplary processes for the prescribing of and administering of prescription drug medication according to exemplary embodiments of the present disclosure;

FIG. 2 is an illustration of illustrative medication packaging that can be used in exemplary embodiments of the present disclosure;

FIG. 3 is a diagram of a process using which a patient can scan QR codes, or other machine-readable optical labels, on prescription medication packaging in accordance with exemplary embodiments of the present disclosure in a manner which reduces and/or minimizes the patient's ability to abuse their prescription medication;

FIG. 4 is a block diagram of a private-by-design SSI architecture implemented by the network and web dashboard in exemplary embodiments of the present disclosure;

FIG. 5 is an illustration of different exemplary user interfaces provided by the web dashboard and patient mobile wallet provided to and communicating between different individuals and entities utilizing exemplary embodiments of the present disclosure;

FIG. 6 is a block diagram of an exemplary embodiment of a system according to the present disclosure;

FIG. 7 is an exemplary network diagram and components of the system according to an exemplary embodiment of the present disclosure; and

FIG. 8 is a block diagram illustrating an exemplary process for issuing and verifying credentials in using an SSI network in the exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

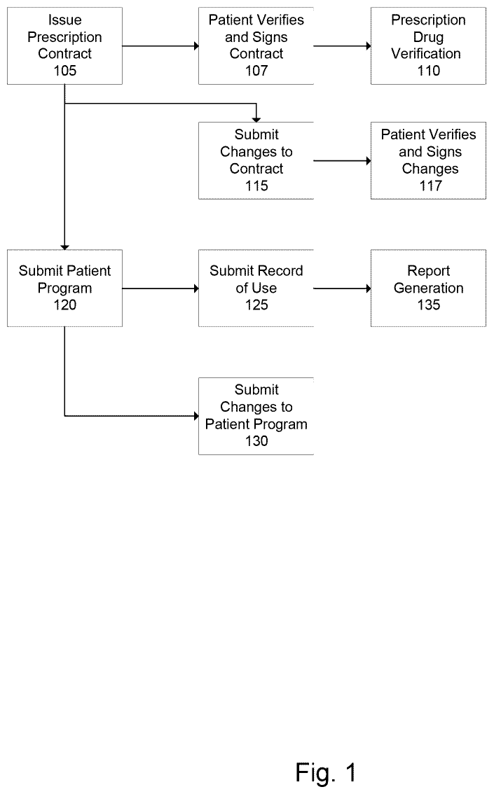

FIG. 1 illustrates a block diagram of exemplary processes for the prescribing and administering of prescription drug medication on a distributed network described in exemplary embodiments of the present disclosure. The exemplary processes shown in a flow diagram of FIG. 1 can be carried out through peer-to-peer, cryptographic interaction between different devices using a SSI architecture as described further herein. The exemplary SSI architecture facilitates the sharing of confidential information while maintaining that only those who are properly credentialed shall receive access to such information

For example, in block 105, using the exemplary system, method and computer-accessible medium, a physician can submit a prescription contract that can include, for example, information identifying a patient, the name and dosage, and quantity of prescription medication, and the digital signature of the physician, to the patient's mobile wallet, via peer-to-peer, cryptographic interaction using a SSI architecture as described herein. As used herein, the term “physician” can refer to any caregiver of the patient, including without limitation, the patient's general care practitioner, psychotherapist, and any other medical practitioner who can prescribe prescription medication to the patient. In block 107, using the exemplary system, method and computer-accessible medium, the patient can verify and sign the contract.

In block 110, using the exemplary system, method and computer-accessible medium, after signing and verifying the contract, a patient can present the prescription contract to a pharmacy which can occur via remote electronic communication between the patient and the pharmacy, such as a peer-to-peer, cryptographic interaction, or by an in person interaction, whereby the patient presents his or her smart phone containing information pertaining to the prescription contract, and the pharmacy can verify the prescription contract based on, for example, the validity of the digital signature of the physician. The interaction between the patient and pharmacy may include the exchange of credentials between the patient and pharmacy using an SSI architecture.

In block 115, using the exemplary system, method and computer-accessible medium, a physician can submit changes to the prescription contract to the patient and/or pharmacies via peer-to-peer, cryptographic interaction using an SSI architecture. The changes to the prescription contract can include a change to the dosage or the type of prescribing medication, and a frequency that the medication can be refilled. Thereafter, in block 117, using the exemplary system, method and computer-accessible medium, the patient can verify and sign the modified contract.

In block 120, using the exemplary system, method and computer-accessible medium, a physician can further submit a patient program, which can include information such as, e.g., an individualized digital schedule for the administration of the prescription medicine, including information specifying when a patient shall take a dose of the prescription medication and procedures of recording use of the prescription medication to the patient and/or other entities, via peer-to-peer, cryptographic interaction using an SSI architecture.

In block 125, using the exemplary system, method and computer-accessible medium, a patient can submit a record of their use of the prescription medication in accordance of their patient program, which can include information such as information scanned from a QR code, or another optically readable code, contained in the packaging of the drug medication, which is described in further detail herein, as well as a time and date information, and information pertaining to other features of the medication such as taste or tongue color, as well as the symptoms of the patient, to the physician and or other entities via peer-to-peer, cryptographic interaction using an SSI architecture.

In block 130, using the exemplary system, method and computer-accessible medium, a physician can submit changes to the patient program to the patient and/or other entities, via peer-to-peer, cryptographic interaction using an SSI architecture. The submission can include information such as a change in when a patient shall take a dose of the prescription medication and a change in the procedure for recording the patient's use of the prescription medication.

In block 135, using the exemplary system, method and computer-accessible medium, one or more reports can be generated and presented on the web dashboard 102 after the submission of at least one record of use to the web dashboard by the patient. The patient may share the reports to the physician and/or other entities via one or more peer-to-peer, cryptographic interactions using an SSI architecture.

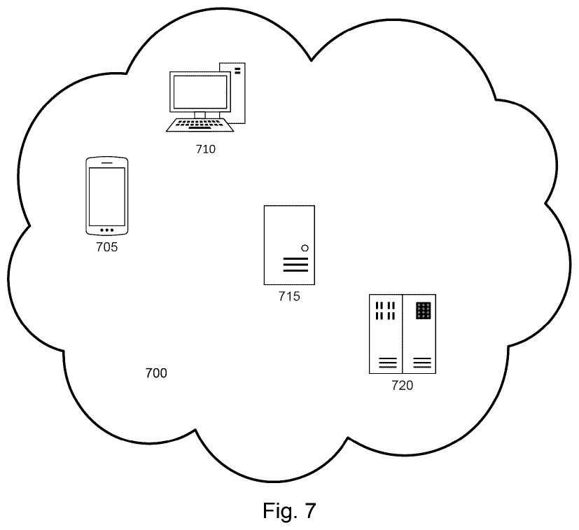

The exemplary process illustrated in FIG. 1 may be carried out on an exemplary network/configuration 700 shown in FIG. 7, which can comprise of a plurality of electronic devices, such as, e.g., computers 705, 710, and related components and equipment 715 and 720, to facilitate the operation of the processes in FIG. 1 to be performed. The equipment shown in FIG. 7 is merely illustrative and not intended to be limiting. Computers 705, 710 can include, for example, the mobile device of a patient 705 who can access the prescription contract described with reference to FIG. 1 on their digital wallet. The exemplary network/configuration 700 can include additional computers and other equipment, for example, computers associated with physician or pharmacies and other entities.

A web dashboard can be provided as an exemplary interface for physicians, pharmacies, pharmaceutical companies, and authorized third parties (such as guardians, family members, and employers of the patient) to manage and perform peer-to-peer interactions in the decentralized SSI network. The web dashboard, as well as patient mobile application can be implemented as a software, as a program or application programming interface (API), on a device (such as computers 705, 710) and provide input, access, and use of prescription contract, patient program, and record of use information stored in the issuer/verifier database, which is further described herein. An App Server or the like may be provided to host the web dashboard contents and data and anything else necessary for computers 705, 710 to execute the web dashboard.

According to additional exemplary embodiments of the present disclosure can provide exemplary mechanisms, whereby a patient can receive and fulfil a prescription contract for their prescription drug through peer-to-peer, cryptographic interactions with their physician. Contract terms of a prescription contract provided as a Verifiable Credential can be exchanged through the issuer/verifier database 715 to facilitate a patient to prove to a physician that they have taken their medication at the prescribed dose and frequency. During the peer-to-peer cryptographic interactions between the physician and patient via the prescription contract facilitated by exemplary embodiments, automated or physician-led actions can be taken to adjust the contract terms. Contract fulfilment validation can be implemented by a digital signature of a physician, and the prescription contract can be presented by the patient to a pharmacy to fulfil the prescription in a peer-to-peer interaction with a pharmacy or other health care site. The validation of the drug prescription can be digitally signed by the pharmacy or care site if the drug prescription is legitimate and the patient can receive a limited amount of medication for remote use with a new prescription contract being initiated at the dispensation of each round of medication.

Using the exemplary system, method and computer-accessible medium according to certain exemplary embodiments of the present disclosure, patients can be restricted to taking their prescribed medication in accordance with an individualized digital schedule contained in their patient's program created by their physician and stored in the patient's mobile wallet. Prior to extracting each pill from a medication package, a patient can be required to undertake certain steps, such as scanning a QR code and entering additional information after the fact, such as, for example the taste, machine-assessed tongue color caused by pill ingestion, and/or the patient's symptoms at the time the medication was taken, their identity, and geographic location. Such additional information can be recorded in the physician web dashboard, and can thereafter be used, for example, in clinical trials, and studies by pharmaceutical companies, or other corporate or governmental members of the medical and pharmaceutical communities.

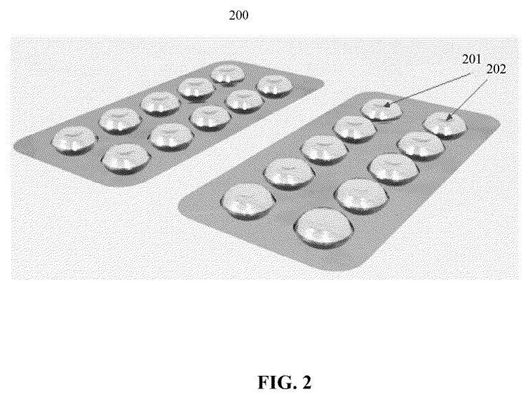

FIG. 2 depicts an illustration of an exemplary medication packaging 200 that can be used in the exemplary embodiments of the present disclosure. The exemplary packaging 200 can include foil on which a QR code can be provided that can be destroyed to access the medication, and which itself conceals additional QR codes that are positioned on the inside of the pill-containing receptacle of the blister pack for each dose 201, 202 of prescription medication contained in the packaging.

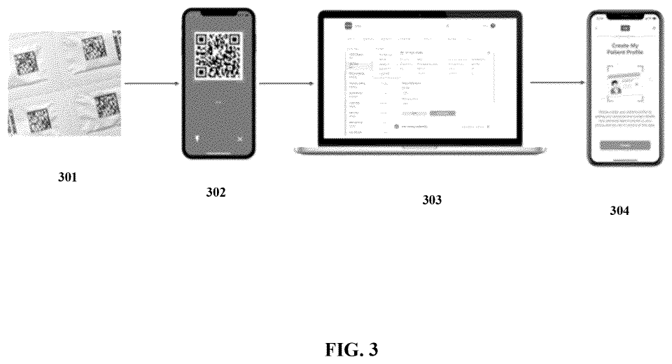

FIG. 3 shows a block diagram of a process by which a patient can scan QR codes, or other machine-readable optical labels, on prescription medication packaging in accordance with the exemplary embodiments of the present disclosure, and enter taste and machine-assessed tongue color caused by pill ingestion in a manner which reduces or minimizes the patient's ability to abuse their prescription medication, including by not taking the medication according to the prescribed schedule. For example, block 301 depicts an example of blister packaging of the prescription medication packaging marked with a tamper proof QR code after a foil is removed. The QR code or other machine-readable optical label can contain a unique identifier of the particular unit in the prescription medication package and other essential product information that can be read out using conventional techniques. Each QR code can be printed, or otherwise disposed of on the prescription medication packaging using ink that is light and/or air sensitive so that the QR code will fade after the foil has been removed from the packaging. This exemplary feature can be used to prevent abuse of the prescription medication because the patient opening all of the pills at once would complicate a patient's ability to scan QR codes in accordance with patient program, and thus would invariably alert the prescribing physician and other entities using the tracking application dashboard of the patient's failure to comply with the patient program.

Patients can use the mobile App to access instructions on how and when to take the prescription medication in accordance with the patient program. Using a smart phone and the patient mobile application a patient can scan the QR code pertaining to the dose of prescription medication taken in block 302 in accordance with the patient program. Using the web dashboard a physician can record and monitor the patient's compliance with the patient program, e.g., the scanning of the QR code by a patient's smart phone can automatically submit a record of use, including, for example, information pertaining to the scanned QR code, a time, geographic location, photograph, third-party attestation, and identity of the patient, to the web dashboard. Such information can provide evidence in support of compliance according to empirical norms established by data aggregated from all users using the web dashboard. The exemplary process, system and computer-accessible medium can advantageously utilize such data in rules-based, machine learning and artificial intelligence algorithms to notify physicians when their patient may be abusing medicine. The patient can also be prompted to describe any symptoms at the time of the record of use by the web dashboard if required by the patient program.

In block 303, the physician can receive the record of use on the web dashboard, which in block 303 can be accessed on a computer. The web dashboard of the physician can be configured to facilitate the physician to verify sensitive personal data contained in the record of use. Such verification can occur by the physician verifying self-attested credentials from the patient in a peer-to-peer interaction as described herein. Further, according to certain exemplary embodiments of the present disclosure, it is possible to provide reports and the like to automatically be generated based on submitted records of use, prescription contracts, patient programs and other health and non-health related data which is described further herein. The physician can access such reports through the web dashboard, and make any changes to the prescription contract, patient program, outreach to the patient through peer-to-peer messaging or surveying and can even cancel prescriptions based on the automatically generated reports and notifications provided on the web dashboard.

In block 304, a patient computer program or prescription contract (e.g., executed by one or more computer processors) can be updated and transmitted to the patient's mobile wallet. A patient can present updates to their prescription contract or patient program to a pharmacy or another health care provider and prescription fulfillment by the pharmacy, for example, or adjustments to care provided by the additional health care providers can occur based on the updates. The presentation and verification of updates by a patient to a pharmacy or additional health care provider can occur remotely or in-person through seamless functionality provided in the patient mobile wallet and the web dashboard or the patient can physically present their smart phone displaying a code to the pharmacy or additional health care provider.

The monitoring of patient compliance with their patient programs can, for example facilitate pharmaceutical companies to make certain changes to the potency of their drugs, and/or recommended dosages thereof, and can facilitate the pharmaceutical companies to learn valuable insights through clinical trials using information recorded in exemplary embodiments. Further, the monitoring of patient compliance with their patient programs can facilitate prescribing physicians and related health care entities to adjust or cancel the patient's prescription. According to certain exemplary embodiments of the present disclosure, it is possible to facilitate such medical decisions using artificial intelligence and analytics using information from patient programs, prescription contracts, and records of use.

Authorized third parties, such as guardians of patients, employers of patients, and/or other third-parties who are on a need-to-know basis, can ask for the patient's consent to be given access to certain patient information, including patient program and prescription contract information, using zero-knowledge proofs via the web dashboard. In cryptography, a zero-knowledge proof protocol can be used so that one party can prove to another party that they know a specific value without conveying any information apart from the fact that they know such a value. The web dashboard can facilitate the use of a zero-knowledge proof protocol by associating provided credentials with a particular role of a user of the web dashboard, and restricting access to information depending on the associated role of the user. In certain exemplary embodiments of the present disclosure, it is possible to use zero-knowledge proof protocol to maintain the patient's privacy while making disclosures to the third-party, without, for example, disclosing medication or medication regimen information of the patient.

In various exemplary embodiments of the present disclosure, one or more reports can be generated and presented in the web dashboard based on at least one interaction between a patient and a physician or other authorized party. The physician can be facilitated to sign one or more report in the web dashboard, and the signature and any changes made to the report by the physician can be transmitted to the patient's mobile wallet. Further, the physician can thereafter adjust the patient's program as needed, as well as provide individualized insights and can transmit such information through the decentralized network via the web dashboard.

The web dashboard can provide different levels of data management and insight, as well as different levels of patient anonymity based on the needs and requirements of the different entities accessing the web dashboard.

Physicians, pharmaceutical companies, and/or other health care providers can interact with the patient and each other in other manners using the web dashboard. For example, such entities can request for the patient to complete a survey, and the survey can be completed by the patient using the patient's mobile wallet. Completion of such surveys can be required as part of a patient's program, or they can be voluntary. They can be completed at a particular frequency or on a one-time basis. The surveys can include questions regarding a patient's symptoms, environmental conditions and so on. The survey requests as well as completed surveys, can be shared through peer-to-peer interaction using the SSI architecture described herein.

According to certain exemplary embodiments of the present disclosure, it is possible to utilize the combined data inputs stored on a physician database (which may include data such as prescription contracts, prescription contract fulfilment, patient programs, records of use, reports, and surveys) with physician support procedures to generate outputs such as: (i) corrective behavioral notification to the patient delivered on the web dashboard; (ii) automatic adjustment to the patient's program, (iii) notifications to the physician or authorized third parties delivered on the web dashboard for a recommended intervention of the patient; and (iv) notifications to the physician delivered on the web dashboard pertaining to a recommendation as to whether the patient is a suitable candidate for a remote prescription plan.

The combination of active stakeholder consent, cryptographically verifiable peer-to-peer interactions, dedicated user interfaces, intelligent automation and material science described in exemplary embodiments of the present disclosure facilitates a robust and private-by-design system of trust that can decrease prescription drug abuse and make medicine predication, prescription fulfilment, and prescription drug administration more efficient. According to certain exemplary embodiments of the present disclosure, it is possible to provide a more transparent, secure and verifiable solution to prescription drug management than the conventional approaches.

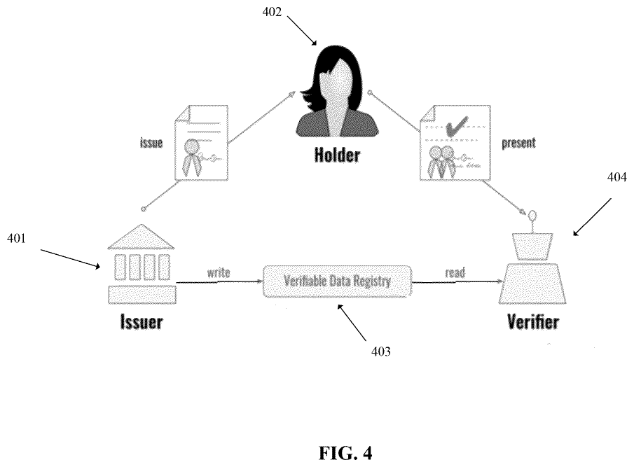

FIG. 4 illustrates a block/flow diagram of a private-by-design SSI architecture according to an exemplary embodiment of the present disclosure. The exemplary architecture can facilitate a trust framework by which any entity accessing information on the decentralized network using the web dashboard may confidently trust that such information is authentic and has not been tampered with.

The exemplary architecture shown in FIG. 4 can employ a flexible and secure communication protocol which facilitates key stakeholders in digital trust transactions to interact via peer-to-peer communication with confidence that information shared in the SSI network is only accessible by those who are authorized to access such information. Such exemplary architecture is merely illustrative, and those of ordinary skill in the art will appreciate that other similar architectures, protocols, technologies, approaches, and the like can be substituted with the ones described herein to achieve similar functionalities.

For example, in SSI systems, users generally control the verifiable credentials that they hold and their consent is required to use those credentials, which reduces the unintentional sharing of user's personal data. Credentials of the entities (i.e. “credential holders” 402) involved in the network (e.g. physicians, patients, pharmaceutical companies, authorized third parties) can be stored in the issuer/verifier database 403 or in another location (e.g. a holder's mobile wallet). A credential issuer 401 can be, for example, an entity which originally issues credentials. A credential holder 402 can be, for example, the patient, physician, pharmaceutical company, or authorized third party. For example, when the holder wishes to access information, the holder can present its credentials to a verifier at block 404, which can verify the credentials. The verifier 404 can be another device on the network, e.g., other than the holder 402.

The exemplary architecture according to the exemplary embodiments of the present disclosure, e.g., as shown in FIG. 4 can include the following benefits: (i) active patient consent can be seamlessly given whenever patients share their data to physicians and third parties; (ii) stakeholders using the architecture can be assured that technology platform providers implementing the architecture cannot use, store or process any of personal or health related data; (iii) data sharing can be completed and verified through zero-knowledge proof or selective disclosure, binding data minimization into every interaction to preserve patient privacy; (iv) verification of data is automated through decentralized public key infrastructure and cryptography, removing the need for intermediaries, APIs and eliminating fraud; and/or (5) peer-to-peer technology can facilitate interactions between patients, physicians, and other shareholders in a fast and intuitive manner, ensuring the adaptability of the system to future requirements.

Further, such exemplary architecture can streamline the accreditation of medical professionals. In conventional processes, turnaround times for issuing digital credentials to medical professionals on and offline is significant, and typically involves reviewing and processing of a great volume of files and data-rich repositories. Moreover, such conventional processes typically require significant human and institutional engagement.

Using the exemplary architecture described herein, the digital credential process for medical professionals can be expedited because after credentials are issued to such medical professionals, a record of their credentials will be stored in the medical professional mobile wallet and verifiable by any third-party, including people and institutions, with whom the medical professional wishes to interact. Once this is established, the medical professional need only present a subset of its credentials to verifiers on the SSI network to establish its identity, and may not have to provide its full medical credentials every time it interacts with stakeholders on the SSI network. Such exemplary implementation for credentialing of medical professionals in the context of digital services and prevention of prescription drug abuse can drastically reduce man-hours and operating costs typically involved with such an application.

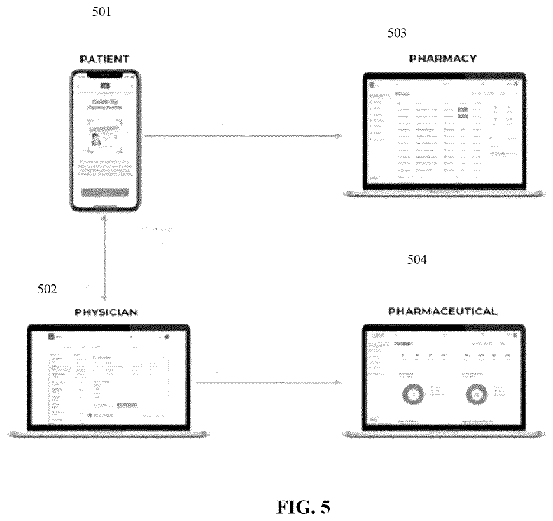

FIG. 5 illustrates a block/flow diagram of an examples of use of different user interfaces provided of the web dashboard provided to different entities utilizing exemplary embodiments of the present disclosure. Illustration 501 shows an example of a user interface of the patient mobile application presented to the patient on a smart phone. Illustration 502 shows an example of a user interface of the web dashboard presented to physician or other caregiver on a computer. Illustration 503 shows an example of a user interface of the web dashboard presented to a pharmacy on a computer. Illustration 504 shows an example of a user interface of the web dashboard presented to a pharmaceutical company on a computer.

Using the exemplary web dashboard and patient mobile application according to various exemplary embodiments of the present disclosure, stakeholders can receive numerous benefits. For example, the patient can receive accurate medical information, manage their prescription medication and prescription regimen through peer-to-peer interactions on the patient mobile application, and by virtues of the features of exemplary embodiments described herein, be less vulnerable to prescription medication abuse. The physician can remotely issue prescription contracts, patient programs and monitor their patients (e.g. their patient's compliance with its patient program) through the web dashboard, and be assured that prescription drug abuse is minimized. Pharmacies can accurately and quickly verify and fulfil prescriptions using the web dashboard, and can participate in efforts to avoid prescription drug abuse by providing feedback to stakeholders on the SSI network pertaining to prescription fulfilment. Further, through the web dashboard pharmaceutical companies and other authorized third-parties can be given access to a plethora of aggregated and fully-, partially- or de-anonymized patient data that can be used for research and development, regulatory processes, sales and marketing, and inventory management.

In order to further prevent abuse of prescription drugs by patients, according to various exemplary embodiments of the present disclosure, it is possible to employ rules-based approaches, machine learning and artificial intelligence techniques to rate patient's propensity to abuse their medication at particular points in time. This advantageously can facilitate early or preventative intervention by the physician or an appointed guardian. Such early intervention can also increase the rate of adherence, reduce the rate of hospitalization, improve the on-going care outcomes for the patient and increase the overall proportion of patients permitted to conduct at-home medication.

Patients who are determined to abuse the drug can do so by Physician Shopping for Prescription Drugs (PSPD). For example, if the patient's refill is declined by their current physician, they will look for a different physician with no prior knowledge of the patient. Exemplary embodiments can advantageously mitigate this risk by implementing an anonymous patient authentication process using a biometric hash in conjunction with a 1-to-many search—i.e. if that hash has been used to authenticate an ID at a different physician in the last 90-days, a flag is raised requiring further inquiry.

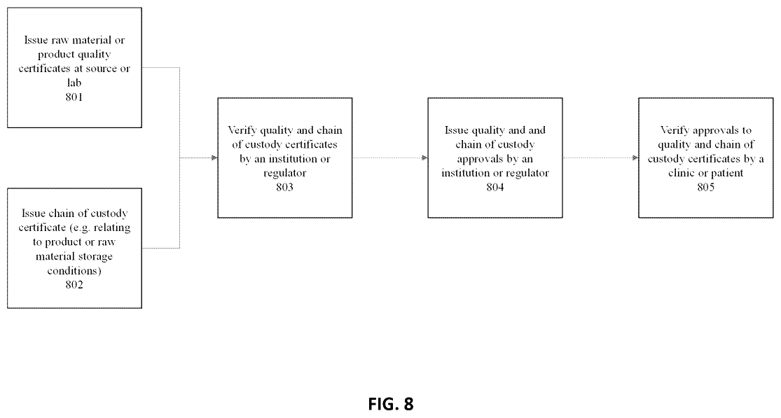

FIG. 8 is a flow diagram illustrating an exemplary process for issuing and verifying credentials in using an SSI network in exemplary embodiments of the present disclosure. For example, in step 801 of FIG. 8, a quality certificate can be issued for a piece of raw material or product pertaining to the quality thereof by a source (e.g. a manufacturer or producer) and/or a laboratory. A laboratory can issue a certificate attesting to the quality of a pack of prescription medication. At step 802, a valid chain of custody certificate relating to, for example, the storage or transit conditions of the raw material or product may be issued. At step 803, an institution or regulator can verify the quality and chain of custody certificates, and at step 804, the institution or regulator can issue approval to the quality and chain of custody certificates. The quality and chain of custody certificates and approvals thereof can be stored in a data registry on an SSI network, as described more fully herein. At step 805, the quality and chain of custody certificate approvals can be verified by a health clinic, patient, or any other end user in a peer-to-peer transaction as described herein. For example, when a clinic receives a prescription medication from a supplier to provide to its customers, the clinic can verify the quality and chain of custody certificates and approval thereof to a verifier (which can be another device on the SSI network) when the clinic acquires the prescription medication.

Exemplary embodiments of the present disclosure can be utilized for the following additional features and advantages to the pharmaceutical, nutraceutical and bioceutical providers:
For example, manufacturers and producers of medicines can establish chain of custody from production through transport of the products to dispensing sites.
By establishing chain of custody, with interactions marked by time and location stamped certifications, it becomes straightforward to establish whether the products have been managed and treated within the limits of the product specifications for atmospheric parameters such as temperature, humidity, and light exposure, as well as document delays as may occur in transport and other logistics, which can affect product quality and integrity.
Manufacturers and producers of medicines may also attest to the quality of their product by certifying the products origin, chemical composition/growing conditions, laboratory or farm credentials, safety, dosing advice, etc.
Other stakeholders such as regulators, authorities, clinics, physicians, psychiatrists, patients, journalists, investors and customers may verify those certificates, for example by scanning a QR code on the packaging with their mobile phone or hand held scanning device. The product owner would be able to assign a core set of information to be verifiable by all stakeholders, plus additional information that relevant to particular stakeholders, e.g. regulators, physicians and patients are provided access to a common set of verifiable data plus whatever is particularly relevant to or required by them as stakeholders.
The interactions as defined herein for patient—physician communications and transactions are not materially different from the perspective of the SSI network and present disclosures, from the interactions that establish and facilitate production, certification, validation, transport, receipt and handling of the products until they are dispensed to patients. As such the present system will be useful for the management of the production of nutraceutical, bioceuticals as well as pharmaceuticals.

Exemplary embodiments of the present disclosure can be utilized for the following additional features and advantages to patients and physicians:
For example, patients can achieve better insight (for example a score accessible to them and their physician on their propensity to comply with the prescription programme), and management of their prescribed drug programs than conventional drug prescription programs, which can facilitate prevention of the prescription drug abuse.
By reducing the likelihood of drug abuse, adherent and compliant patients can enjoy the convenience and comfort of at home prescription plans instead of having to regularly visit a care site for medication dispensation or physician assessments. In the same vein, physicians can expect a more efficient and effective relationship with such patients
The lack of knowledge that often leads to prescribed drug abuse can be addressed by providing the relevant information and individualized insights to the patient and physician.
A “3-in-1” solution of drugs, digital monitoring of drug use and personalization of the patient's program can be provided while limiting the abuse of the prescription drug.
Individual dose identification can be provided to physicians and other medical care providers to prevent counterfeiting and medication errors
Prescription drug abuse by patients is addressed and patient's compliance with plans designed by practitioners will increase, as will the patient's satisfaction.
Individual risk factors (such as past or present addictions to other substances, including alcohol and tobacco; family history of substance abuse problems; certain pre-existing psychiatric conditions; exposure to peer pressure or a social environment where there is drug use) can be determined and monitored by prescribing physicians and other medical care providers.

Personalized medicine and follow-on sales can be provided to patients to facilitate prescription management, submission of symptom reports, dosing reminders insight generation, and adjustment to the patient programs.
Use data can be applied as evidence to support future drug use and prescriptions, for example by enabling physicians and care sites to record and prove the month-on-month change in prescription compliance of their patients.
Use data can be used to generate billing and invoicing for health-related activities.
Patients can compile, store, recollect, share and review medical records, as well as share those records with third parties of their choosing on the SSI network, possibly in return for remuneration, which may include researchers, caregivers, insurance companies and other parties that may be easily added to SSI network as stakeholders.
Caregivers in the SSI network may refer patients to one another where relevant, for example by inviting a caregiver to the SSI network and issuing the patient with a “referral attestation” linked to a particular caregiver, who may request the relevant health record data for their care provision directly from the patient.

According to further exemplary embodiments of the present disclosure, the following additional features and advantages to pharmaceuticals developers and manufacturers can be provided:
Matching anonymized or de-anonymized adherence, compliance, side-effect, demographic and environmental data received from patients to a specific drug can assist with R&D.
Clinical trial consent gathering, data submission and support activities can enhance R&D and demonstrate that a certain prescription drug can be used remotely in a disciplined way that allows personalized adjustments of the patient medication program.
Facilitating for easier and faster approvals by enabling the use of data for monitoring compliance and non-abuse.
Facilitating an early intervention that can prevent the problem from turning into an addiction and major social problem.
Generating verifiable inventory reports from customers and care sites alongside dosing schedules in different markets facilitates active inventory management.
Demand-side data can be used to inform models that predict peaks and troughs in demand in different markets and support manufacturing planning.

It can be appreciated by those persons skilled in the art that the various embodiments described herein are capable of broad utility and application. Accordingly, while the various embodiments are described herein in detail in relation to the exemplary embodiments, it is to be understood that this disclosure is illustrative and exemplary of the various embodiments and is made to provide an enabling disclosure. Accordingly, the disclosure is not intended to be construed to limit the embodiments or otherwise to exclude any other such embodiments, adaptations, variations, modifications and equivalent arrangements.

The system described above can be implemented with servers and other computing devices in various configurations. The various servers and computing devices can use software to execute programs to execute the methods described above. Various exemplary embodiments of the present disclosure can also relate to the software or computer readable medium containing program instructions for executing the above described methods.

Although the foregoing examples show the various exemplary embodiments of the present disclosure can in one physical configuration, it is to be appreciated that the various components can be located at distant portions of a distributed network, such as a local area network, a wide area network, a telecommunications network, an intranet and/or the Internet. Thus, it should be appreciated that the components of the various embodiments can be combined into one or more devices, collocated on a particular node of a distributed network, or distributed at various locations in a network, for example. As will be appreciated by those skilled in the art, the components of the various embodiments can be arranged at any location or locations within a distributed network without affecting the operation of the respective system.

Communications networks connect the various computing devices described above and can be comprised of, or can interface to any one or more of, for example, the Internet, an intranet, a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1 or E3 line, a Digital Data Service (DDS) connection, a Digital Subscriber Line (DSL) connection, an Ethernet connection, an Integrated Services Digital Network (ISDN) line, a dial-up port such as a V.90, a V.34 or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The communications networks that connect the various computing devices described above can also comprise, include or interface to any one or more of a Wireless Application Protocol (WAP) link, a Wi-Fi link, a microwave link, a General Packet Radio Service (GPRS) link, a Global System for Mobile Communication (GSM) link, a Code Division Multiple Access (CDMA) link or a Time Division Multiple Access (TDMA) link such as a cellular phone channel, a GPS link, a cellular digital packet data (CDPD) link, a Research in Motion, Limited (RIM) duplex paging type device, a Bluetooth radio link, or an IEEE 802.11-based radio frequency link. Communications networks can further comprise, include or interface to any one or more of an RS-232 serial connection, an IEEE-1394 (Firewire) connection, a Fiber Channel connection, an infrared (IrDA) port, a Small Computer Systems Interface (SCSI) connection, a Universal Serial Bus (USB) connection or another wired or wireless, digital or analog interface or connection.

In some embodiments, the communication networks can comprise a satellite communications network, such as a direct broadcast communication system (DBS) having the requisite number of dishes, satellites and transmitter/receiver boxes, for example. The communications network can also comprise a telephone communications network, such as the Public Switched Telephone Network (PSTN). In another exemplary embodiment, communication networks can comprise a Personal Branch Exchange (PBX), which can further connect to the PSTN.

Although examples of servers and personal computing devices are described above, exemplary embodiments of the present disclosure can utilize other types of communication devices whereby a user can interact with a network that transmits and delivers data and information used by the various systems and methods described herein. The personal computing devices can include desktop computers, laptop computers, tablet computers, smart phones, and other mobile computing devices, for example. The servers and personal computing devices can include a microprocessor, a microcontroller or other device operating under programmed control. These devices can further include an electronic memory such as a random access memory (RAM), electronically programmable read only memory (EPROM), other computer chip-based memory, a hard drive, or other magnetic, electrical, optical or other media, and other associated components connected over an electronic bus, as will be appreciated by persons skilled in the art. The personal computing devices can be equipped with an integral or connectable liquid crystal display (LCD), electroluminescent display, a light emitting diode (LED), organic light emitting diode (OLED) or another display screen, panel or device for viewing and manipulating files, data and other resources, for instance using a graphical user interface (GUI) or a command line interface (CLI). The personal computing devices can also include a network-enabled appliance or another TCP/IP client or other device. The personal computing devices can include various connections such as a cell phone connection, Wi-Fi connection, Bluetooth connection, satellite network connection, and/or near field communication (NFC) connection, for example.

The servers and personal computing devices described above can include at least one programmed processor and at least one memory or storage device. The memory can store a set of instructions. The instructions can be either permanently or temporarily stored in the memory or memories of the processor. The set of instructions can include various instructions that perform a particular task or tasks, such as those tasks described above. Such a set of instructions for performing a particular task can be characterized as a program, software program, software application, app, or software. The modules described above can comprise software, firmware, hardware, or a combination of the foregoing.

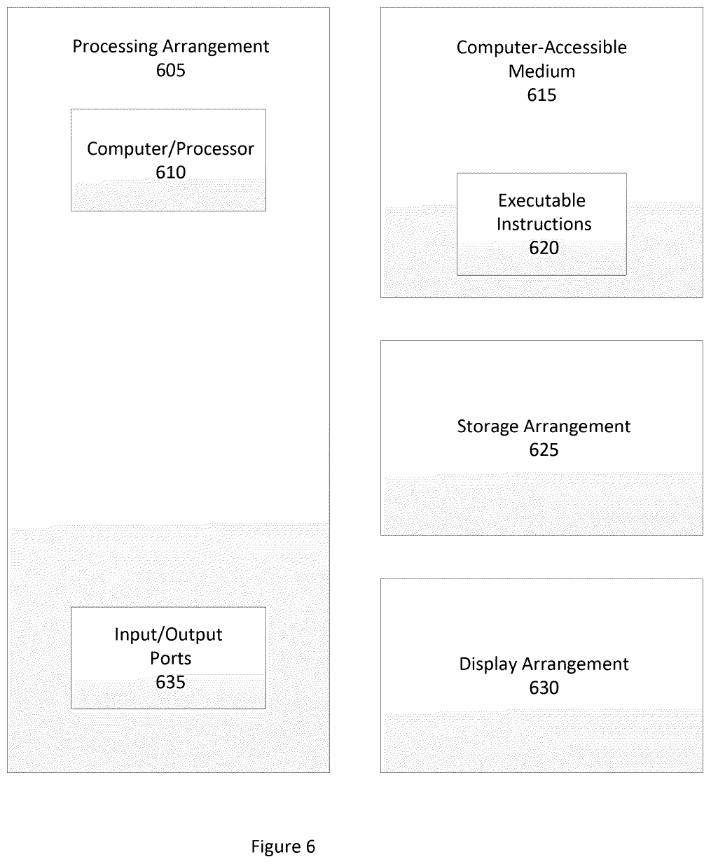

FIG. 6 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 605. Such processing/computing arrangement 605 can be, for example entirely or a part of, or include, but not limited to, a computer/processor 610 that can include, for example one or more microprocessors, and use instructions stored on a computer-accessible medium (e.g., RAM, ROM, hard drive, or other storage device).

As shown in FIG. 6, for example, a computer-accessible medium 615 (e.g., as described herein above, a storage device such as a hard disk, floppy disk, memory stick, CD-ROM, RAM, ROM, etc., or a collection thereof) can be provided (e.g., in communication with the processing arrangement 605). The computer-accessible medium 615 can contain executable instructions 620 thereon. In addition or alternatively, a storage arrangement 625 can be provided separately from the computer-accessible medium 615, which can provide the instructions to the processing arrangement 605 so as to configure the processing arrangement to execute certain exemplary procedures, processes, and methods, as described herein above, for example.

Further, the exemplary processing arrangement 605 can be provided with or include an input/output ports 635, which can include, for example a wired network, a wireless network, the internet, an intranet, a data collection probe, a sensor, etc. As shown in FIG. 6, the exemplary processing arrangement 605 can be in communication with an exemplary display arrangement 630, which, according to certain exemplary embodiments of the present disclosure, can be a touch-screen configured for inputting information to the processing arrangement in addition to outputting information from the processing arrangement, for example. Further, the exemplary display arrangement 630 and/or a storage arrangement 625 can be used to display and/or store data in a user-accessible format and/or user-readable format.

It is appreciated that in order to practice the methods of the embodiments as described above, it is not necessary that the processors and/or the memories be physically located in the same geographical place. That is, each of the processors and the memories used in exemplary embodiments of the present disclosure can be located in geographically distinct locations and connected so as to communicate in any suitable manner. Additionally, it is appreciated that each of the processor and/or the memory can be composed of different physical pieces of equipment. Accordingly, it is not necessary that the processor be one single piece of equipment in one location and that the memory be another single piece of equipment in another location. That is, it is contemplated that the processor can be two or more pieces of equipment in two or more different physical locations. The two distinct pieces of equipment can be connected in any suitable manner. Additionally, the memory can include two or more portions of memory in two or more physical locations.

As described above, a set of instructions is used in the processing of various embodiments of the present disclosure. The servers and personal computing devices described above can include software or computer programs stored in the memory (e.g., non-transitory computer readable medium containing program code instructions executed by the processor) for executing the methods described herein. The set of instructions can be in the form of a program or software or app. The software can be in the form of system software or application software, for example. The software might also be in the form of a collection of separate programs, a program module within a larger program, or a portion of a program module, for example. The software used might also include modular programming in the form of object oriented programming. The software tells the processor what to do with the data being processed.

Further, it is appreciated that the instructions or set of instructions used in the implementation and operation of the present disclosure can be in a suitable form such that the processor can read the instructions. For example, the instructions that form a program can be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processor, i.e., to a particular type of computer, for example. Any suitable programming language can be used in accordance with the various embodiments of the present disclosure. For example, the programming language used can include assembly language, Ada, APL, Basic, C, C++, COBOL, dBase, Forth, Fortran, Java, Modula-2, Pascal, Prolog, REXX, Visual Basic, and/or JavaScript and others. Further, it is not necessary that a single type of instructions or single programming language be utilized in conjunction with the operation of the system and method of the present disclosure. Rather, any number of different programming languages can be utilized as is necessary or desirable.

Additionally, the instructions and/or data used in the practice of various exemplary embodiments of the present disclosure can utilize any compression or encryption technique or algorithm, as can be desired. An encryption module might be used to encrypt data. Further, files or other data can be decrypted using a suitable decryption module, for example.

The software, hardware and services described herein can be provided utilizing one or more cloud service models, such as Software-as-a-Service (SaaS), Platform-as-a-Service (PaaS), and Infrastructure-as-a-Service (IaaS), and/or using one or more deployment models such as public cloud, private cloud, hybrid cloud, and/or community cloud models.

In the systems and methods according to exemplary embodiments of the present disclosure, a variety of “user interfaces” can be utilized to allow a user to interface with the personal computing devices. As used herein, a user interface can include any hardware, software, or combination of hardware and software used by the processor that allows a user to interact with the processor of the communication device. A user interface can be in the form of a dialogue screen provided by an app, for example. A user interface can also include any of touch screen, keyboard, voice reader, voice recognizer, dialogue screen, menu box, list, checkbox, toggle switch, a pushbutton, a virtual environment (e.g., Virtual Machine (VM)/cloud), or any other device that allows a user to receive information regarding the operation of the processor as it processes a set of instructions and/or provide the processor with information. Accordingly, the user interface can be any system that provides communication between a user and a processor. The information provided by the user to the processor through the user interface can be in the form of a command, a selection of data, or some other input, for example.

Although the exemplary embodiments of the present disclosure have been described herein in the context of a particular implementation in a particular environment for a particular purpose, those skilled in the art will recognize that its usefulness is not limited thereto and that the embodiments of the present disclosure can be beneficially implemented in other related environments for similar purposes.

1. A computer implemented method for at least one of creating, sharing or co-signing one or more prescription contracts using verifiable Self-Sovereign Identity (SSI) credentials, comprising:
providing the one or more prescription contracts associated with a medical professional to a mobile storage configuration of a patient via a first digital communication;
submitting a co-execution of the one or more prescription contracts by the patient to a control application of the medical professional via a second electronic communication; and
providing one or more prescriptions associated with the one or more prescription contracts by the patient to a medical product, a device or a medication for a validation via a third electronic communication, wherein in the first, second, and third communications, credentials of at least one of the patient, the medical professional, or the dispensary receiving the one or more prescription contracts or the one or more prescriptions is retrieved and verified using (i) the SSI credentials and (ii) a database which is at least one of an issuer database or a verifier database.

2. The method of claim 1, further comprising:
submitting an electronic program of the patient to the mobile storage configuration via a fourth communication, the electronic program including a digital schedule defining procedures for the submission of an electronic record regarding use of the device, the medication or the product associated with the prescription by the patient; and
submitting the electronic record regarding use of the device, the medication or the product associated with the prescription by the patient to the medical professional via a fifth communication, wherein credentials of at least one of the patient or the medical professional who receive data is retrieved and verified using the database via the fourth and fifth communications.

3. The method of claim 1, further comprising:
modifying the one or more prescription contracts;
submitting the modified one or more prescription contracts by the medical professional to the mobile storage configuration of the patient via a sixth communication;
modifying an electronic program of the patient; and
submitting the modified electronic program by the medical professional to the mobile storage configuration in a seventh communication, wherein credentials of at least one of the patient or the medical professional who receive data is retrieved and verified using the database via the sixth communication.

4. The method of claim 1, further comprising submitting an electronic program of the patient to the mobile storage configuration, the electronic program including a digital schedule defining procedures for the submission of the electronic record regarding use of the medication that includes one or more pills associated with the prescription by the patient, wherein each of the one or more pills includes an individual scannable code, and wherein the digital schedule defines procedures for a submission of the electronic record of use for each of the one or more pills.

5. The method of claim 4, wherein the scannable code for each of the one or more pills is accessible only after a packaging is opened and each of the one or more pills is removed from the packaging.

6. The method of claim 5, wherein the scannable code is composed of an ink that is sensitive to at least one of light or air such that the scannable code fades when the packaging revealing the scannable code is opened.

7. The method of claim 5, wherein the packaging contains a first pill and a second pill of the medication, wherein the first pill includes a first type of ingestible dye, wherein the second pill contains a second type of ingestible dye, wherein a consumption of the first and second pills causes discoloration of a tongue of a patient over a particular time period, wherein the submission of the electronic record associated with a use of the first and second pills includes a submission of a digital photograph of the tongue of the patient after each of the first and second pills is consumed, and wherein a compliance with the digital schedule includes monitoring of the tongue of the patient in submitted digital photographs.

8. The method of claim 5, wherein the packaging contains a plurality of pills, each of the pills having a different taste from another one of the pills, wherein the submission of the electronic record associated with a use of the pills includes a submission of the taste of each of the pills and a scannable code identifying a particular one of the pills ingested, and wherein a compliance with the digital schedule includes a determination of whether the identified taste matches the identified one of the pills.

9. The method of claim 2, wherein the submission of the electronic record regarding the use includes at least one of a geography location, a timestamp, an identity of the patient, a photograph, a third-party attestation, or other meta data.

10. The method of claim 2, further comprising electronically accessing one of the one or more prescription contracts and the electronic program of the patient by a third party using a zero-knowledge proof.

11. The method of claim 2, further comprising communicating with an electronic dashboard for providing access, submission, and verification of the one or more prescription contracts, the electronic record of use and the electronic program of the patient by at least one of the medical professional, the patient, the dispensary or an authorized third party via at least one computer processor.

12. The method of claim 11, wherein the retrieval and verification occurs in a decentralized public key infrastructure.

13. The method of claim 11, wherein the one or more prescription contracts includes confidential information of the patient and wherein the electronic record associated with the use and the electronic program of the patient are restricted from access.

14. A computer-accessible medium which having stored thereon a computer-executable instructions for at least one of creating, sharing or co-signing one or more prescription contracts using verifiable Self-Sovereign Identity (SSI) credentials, wherein, when a computer processor executes the computer-executable instructions, the computer processor is configured to perform procedures comprising:
providing the one or more prescription contracts associated with a medical professional to a mobile storage configuration of a patient via a first digital communication;
submitting a co-execution of the one or more prescription contracts by the patient to a control application of the medical professional via a second electronic communication; and
providing one or more prescriptions associated with the one or more prescription contracts by the patient to a medical product, a device or a medication for a validation via a third electronic communication, wherein in the first, second, and third communications, credentials of at least one of the patient, the medical professional, or the dispensary receiving the one or more prescription contracts or the one or more prescriptions is retrieved and verified using (i) the SSI credentials and (ii) a database which is at least one of an issuer database or a verifier database.

15-26. (canceled)

27. A system for at least one of creating, sharing or co-signing one or more prescription contracts using verifiable Self-Sovereign Identity (SSI) credentials, comprising:
a computer hardware arrangement configured to:
provide the one or more prescription contracts associated with a medical professional to a mobile storage configuration of a patient via a first digital communication;
facilitate a submission of a co-execution of the one or more prescription contracts by the patient to a control application of the medical professional via a second electronic communication; and
provide one or more prescriptions associated with the one or more prescription contracts by the patient to a medical product, a device or a medication for a validation via a third electronic communication,
wherein in the first, second, and third communications, credentials of at least one of the patient, the medical professional, or the dispensary receiving the one or more prescription contracts or the one or more prescriptions is retrieved and verified using (i) the SSI credentials and (ii) a database which is at least one of an issuer database or a verifier database.

28. The system of claim 27, wherein the computer hardware arrangement is further configured to:
facilitate a submission of an electronic program of the patient to the mobile storage configuration via a fourth communication, the electronic program including a digital schedule defining procedures for the submission of an electronic record regarding use of the device, the medication or the product associated with the prescription by the patient; and
facilitate a submission of the electronic record regarding use of the device, the medication or the product associated with the prescription by the patient to the medical professional via a fifth communication, wherein credentials of at least one of the patient or the medical professional who receive data is retrieved and verified using the database via the fourth and fifth communications.

29. The system of claim 27, wherein computer hardware arrangement is further configured to:
facilitate a modification of the one or more prescription contracts;
facilitate a submission of the modified one or more prescription contracts by the medical professional to the mobile storage configuration of the patient via a sixth communication;
modify an electronic program of the patient; and
facilitate a submission of the modified electronic program by the medical professional to the mobile storage configuration in a seventh communication, wherein credentials of at least one of the patient or the medical professional who receive data is retrieved and verified using the database via the sixth communication.

30. The system of claim 27, wherein the computer hardware arrangement is further configured to facilitate a submission of an electronic program of the patient to the mobile storage configuration, the electronic program including a digital schedule defining procedures for the submission of the electronic record regarding use of the medication that includes one or more pills associated with the prescription by the patient, and wherein each of the one or more pills includes an individual scannable code, and wherein the digital schedule defines procedures for a submission of the electronic record of use for each of the one or more pills.

31. The system of claim 30, wherein the scannable code for each of the one or more pills is accessible only after a packaging is opened and each of the one or more pills is removed from the packaging, and wherein at least one of:
the scannable code is composed of an ink that is sensitive to at least one of light or air such that the scannable code fades when the packaging revealing the scannable code is opened,
the packaging contains a first pill and a second pill of the medication, the first pill includes a first type of ingestible dye, and the second pill contains a second type of ingestible dye, wherein a consumption of the first and second pills causes discoloration of a tongue of a patient over a particular time period, wherein the submission of the electronic record associated with a use of the first and second pills includes a submission of a digital photograph of the tongue of the patient after each of the first and second pills is consumed, and wherein a compliance with the digital schedule includes monitoring of the tongue of the patient in submitted digital photographs, and
the packaging contains a plurality of pills, each of the pills having a different taste from another one of the pills, wherein the submission of the electronic record associated with a use of the pills includes a submission of the taste of each of the pills and a scannable code identifying a particular one of the pills ingested, and wherein a compliance with the digital schedule includes a determination of whether the identified taste matches the identified one of the pills.

32-34. (canceled)

35. The system of claim 28, wherein at least one of:
the submission of the electronic record regarding the use includes at least one of a geography location, a timestamp, an identity of the patient, a photograph, a third-party attestation, or other meta data,
the computer hardware arrangement is further configured to facilitate an electronic assessment of one of the one or more prescription contracts and the electronic program of the patient by a third party using a zero-knowledge proof, or
the computer hardware arrangement is further configured to facilitate a communication with an electronic dashboard for providing access, submission, and verification of the one or more prescription contracts, the electronic record of use and the electronic program of the patient by at least one of the medical professional, the patient, the dispensary or an authorized third party via at least one computer processor.

36-39. (canceled)
